(19) United States
(12) Patent Application Publication
(10) Pub. No.: US 2003/0144505 A1
(43) Pub. Date: Jul. 31, 2003

(54) Novel compounds

(75) Inventors: Steven Mark Bromidge, Sawbridgewroth (GB); Francis David King, Bishops Stortford (GB); Paul Adrian Wyman, Epping (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham p.l.c.

(21) Appl. No.: 10/345,499

(22) Filed: Jan. 16, 2003

Related U.S. Application Data
(63) Continuation of application No. 09/850,630, filed on May 4, 2001, now abandoned.
Application No. 09/850,630 is a continuation of application No. 09/666,957, filed on Sep. 20, 2000, now abandoned.
Application No. 09/666,957 is a continuation of application No. 09/331,353, filed on Jun. 18, 1999, now abandoned.
Application No. 09/331,353 is a 371 of international application No. PCT/EP97/07160, filed on Dec. 15, 1997.

(30) Foreign Application Priority Data
Dec. 19, 1996 (GB) 9626376.9
Jan. 17, 1997 (GB) 9700902.1

(51) Int. Cl. C07D 279/12; C07D 265/30; C07D 213/56; C07D 209/14; C07D 241/04

(52) U.S. Cl. 544/59; 544/167; 544/402; 546/336; 548/503; 564/161

(57) ABSTRACT

A compound of formula (I) or a pharmaceutically acceptable salt thereof:

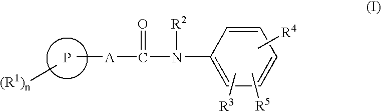

wherein:

P is phenyl, naphthyl, thiophene or benzothiophene;

A is a single bond, a C₁₋₆alkylene or a C₁₋₆alkenylene group;

R¹ is halogen, C₁₋₆alkyl, optionally substituted by one or more halogen atoms, C₃₋₆cycloalkyl, C₁₋₆alkoxy, OCF₃, hydroxy, hydroxyC₁₋₆alkyl, hydroxyC₁₋₆alkoxy, C₁₋₆alkoxyC₁₋₆alkoxy, C₁₋₆alkanoyl, nitro, amino, C₁₋₆alkylamino, di C₁₋₆alkylamino or cyano; or R¹ is phenyl];

n is 0,1, 2, 3, 4, 5 or 6:

R² is C₁₋₆ alkyl phenyl-C₁₋₆alkyl or naphthyl-C₁₋₆ alkyl;

R³ is a group R⁵ or together with R⁵ forms a group (CH₂)₂O or (CH₂)₃O;

R⁴ is a piperazine ring optionally substituted by C₁₋₆ alkyl; and

R⁵ is hydrogen, halogen, C₁₋₆alkyl, C₃₋₆cycloalkyl, C₁₋₆alkoxy, hydroxy, hydroxyC₁₋₆alkyl, hydroxyC₁₋₆alkoxy, C₁₋₆alkoxyC₁₋₆alkoxy, C₁₋₆alkanoyl, nitro, trifluoromethyl, cyano phenyl or naphthyl, is provided. The present compounds are useful as 5-HT₆ receptor modulators..

This invention relates to compounds having pharmacological activity, processes for their preparation, to compositions containing them and to their use in the treatment of CNS disorders.

EPA 0 021 580 and EPA 0 076 072 describe sulphonamide derivatives which are disclosed as having antiarrhythmic activity. A structurally distinct class of compounds has now been discovered, which have been found to have 5HT₆ receptor antagonist activity. 5HT₆ receptor antagonists are believed to be of potential use in the treatment of certain CNS disorders such as anxiety, depression, epilepsy, obsessive compulsive disorders, migraine, Alzheimers disease (enhancement of cognitive memory), sleep disorders, feeding disorders such as anorexia and bulimia, panic attacks, withdrawal from drug abuse such as cocaine, ethanol, nicotine and benzodiazepines, schizophrenia, and also disorders associated with spinal trauma and/or head injury such as hydrocephalus. Compounds of the invention are also expected to be of use in the treatment of certain GI (gastrointestinal) disorders such as IBS (Irritable Bowel Syndrome).

The present invention therefore provides, in a first aspect, a compound of formula (I) or a salt thereof:

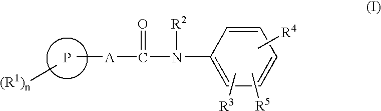

wherein:

P is phenyl, naphthyl, a bicyclic heterocyclic ring or is a 5 to 7-membered heterocyclic ring each containing 1 to 4 heteroatoms selected from oxygen, nitrogen or sulphur;

A is a single bond, a C₁₋₆alkylene or a C₁₋₆alkenylene group;

R¹ is halogen, C₁₋₆alkyl optionally substituted by one or more halogen atoms, C₃₋₆cycloalkyl, COC₁₋₆alkyl, C₁₋₆alkoxy, OCF₃, hydroxy, hydroxyC₁₋₆alkyl, hydroxyC₁₋₆alkoxy, C₁₋₆alkoxyC₁₋₆alkoxy, C₁₋₆alkanoyl, nitro, amino, C₁₋₆alkylamino or diC₁₋₆alkylamino, cyano or R¹ is phenyl, naphthyl, a bicyclic heterocyclic ring or is a 5 to 7-membered heterocyclic ring each containing 1 to 4 heteroatoms selected from oxygen, nitrogen or sulphur;

n is 0, 1, 2, 3, 4, 5 or 6:

R² is C₁₋₆ alkyl or aryl C₁₋₆ alkyl;

R³ is a group R⁵ or together with R⁵ forms a group (CH₂)₂O or (CH₂)₃O or R³ is linked to R² to form a group (CH₂)₂ or (CH₂)₃;

R⁴ is —X(CH₂)p-R⁶ where X is a single bond, CH₂, O, NH or N—C₁₋₆alkyl and p is 0 to 6 and R⁶ is an optionally substituted 5- to 7-membered heterocyclic ring containing 1 to 3 heteroatoms selected from nitrogen, sulphur or oxygen, or R⁶ is NR⁷R⁸ where R⁷ and R⁸ are independently hydrogen, C₁₋₆alkyl or aryl C₁₋₆ alkyl; and

R⁵ is hydrogen, halogen, C₁₋₆alkyl, C₃₋₆cycloalkyl, COC₁₋₆alkyl, C₁₋₆alkoxy, hydroxy, hydroxyC₁₋₆alkyl, hydroxyC₁₋₆alkoxy, C₁₋₆alkoxyC₁₋₆alkoxy, C₁₋₆alkanoyl, nitro, trifluoromethyl, cyano or aryl.

C₁₋₆ Alkyl groups, whether alone or as part of another group, may be straight chain or branched. As used herein the term aryl includes phenyl and naphthyl.

When P is a bicyclic heterocyclic ring suitable examples include benzothiophene, quinoline or isoquinoline. Suitable 5 to 7-membered heterocyclic rings include thienyl, furyl, pyrrolyl, triazolyl, diazolyl, imidazolyl, oxazolyl, thiazolyl, oxadiazolyl, isothiazolyl, isoxazolyl, thiadiazolyl, pyridyl, pyrimidyl, pyrrolidinyl and pyrazinyl. The heterocyclic rings can be linked to the remainder of the molecule via any suitable carbon atom or, when present, a nitrogen atom. Suitable substituents for these rings include R⁵ groups as defined above.

Preferably P is phenyl, thiophene, benzothiophene or naphthyl.

Preferably A is a single bond, an ethyl or —CH═CH— group. Most preferably A is a single bond.

When R¹ is a 5-7 membered heterocyclic or bicyclic heterocyclic ring suitable examples include those given within the description of group P. Preferably R¹ is halogen or C₁₋₄ alkyl optionally substituted by one or more halogens, for example methyl or CF₃.

Preferably n is 0, 1, 2 or 3, particularly 1 or 2.

Preferably R² is C₁₋₆ alkyl, in particular methyl or ethyl.

Suitably R³ is a group R⁵ or together with R⁵ forms a group (CH₂)₂O or (CH₂)₃O or R³ is linked to R² to form a group (CH₂)₂ or (CH₂)₃. It will be appreciated that when R³/R⁵ groups are linked together the two groups must be attached to adjacent carbon atoms of the phenyl ring. Preferably R³ is a group R⁵ in particular hydrogen.

Preferably R⁴ is meta with respect to the carboxamide linkage. Preferably X is a bond, p is 0 and R⁶ is an optionally substituted 5- to 7-membered heterocyclic ring. The heterocyclic rings can be linked to the remainder of the molecule via a carbon atom or, when present, a nitrogen atom. Optional substituents for these rings, which can be present on carbon and/or nitrogen atoms, include C₁₋₆alkyl, in particular methyl. More preferably R⁴ is an optionally substituted piperazine. Most preferably R⁴ is N-methylpiperazine or piperazine.

Preferably R⁵ is C₁₋₆alkoxy, most preferably methoxy. Preferably R⁵ is para with respect to the amide group.

Particular compounds of the invention include:

N-[4-Methoxy-3-(4-methylpiperazin-1-yl)phenyl]-N-methyl-4-phenylbenzamide, 4-Bromo-N-ethyl-N-[4-methoxy-3-(4-methylpiperazin-1-yl)phenyl]-3-methylbenzamide,

4-Bromo-N-[4-methoxy-3-(4-methylpiperazin-1-yl)phenyl]-3-methyl-N-propylbenzamide,

4-Bromo-N-[4-methoxy-3-(4-methylpiperazin-1-yl)phenyl]-3,N-dimethylbenzamide, Naphthalene-2-carboxylic acid N-[4-methoxy-3-(4-methylpiperazin-1-yl)phenyl]-N-methyl amide,

3-Chlorobenzo[b]thiophene-2-carboxylic acid-N-[4-methoxy-3-(4-methylpiperazin-1-yl)phenyl]-N-methyl amide,

3-Bromo-N-[4-methoxy-3-(4-methylpiperazin-1-yl)phenyl]-N-methylbenzamide,

3,4-Dichloro-N-[4-methoxy-3-(4-methylpiperazin-1-yl)phenyl]-N-methylbenzamide,

3-Bromothiophene-2-carboxylic acid-N-[4-methoxy-3-(4-methylpiperazin-1-yl)phenyl]-N-methyl amide,

4-tert Butyl-N-[4-methoxy-3-(4-methylpiperazin-1-yl)phenyl]-N-methylbenzamide,

4-Bromo-N-[4-methoxy-3-(4-methylpiperazin-1-yl)phenyl]-N-methylbenzamide,

3,4-Dichloro-N-(4-methoxy-3-piperazin-1-ylphenyl)-N-methylbenzamide,

3-Chlorobenzo[b]thiophene-2-carboxylic acid N-(4-methoxy-3-piperazin-1-ylphenyl)-N-methyl amide

and pharmaceutically acceptable salts thereof.

The compounds of the formula (I) can form acid addition salts with acids, such as conventional pharmaceutically acceptable acids, for example maleic, hydrochloric, hydrobromic, phosphoric, acetic, fumaric, salicylic, citric, lactic, mandelic, tartaric and methanesulphonic.

Compounds of formula (I) may also form solvates such as hydrates, and the invention also extends to these forms. When referred to herein, it is understood that the term ‘compound of formula (I)’ also includes these forms.

Certain compounds of formula (I) are capable of existing in stereoisomeric forms including diastereomers and enantiomers and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

The present invention also provides a process for the preparation of a compound of formula (I) or a pharmaceutically acceptable salt thereof, which process comprises the coupling of a compound of formula (II):

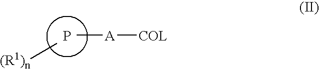

in which R¹, n, P, and A are as defined in formula (I) or protected derivatives thereof and L is a leaving group with a compound of formula (III):

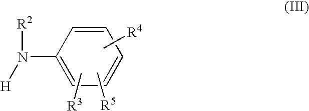

in which R², R³, R⁴ and R⁵ are as defined in formula (I) or protected derivatives thereof and optionally thereafter:

removing any protecting groups,

forming a pharmaceutically acceptable salt.

Suitable leaving groups include halogen, in particular chloro. The reaction of a compounds of formulae (II) and (III) is carried out by mixing the two reagents together, optionally in an inert solvent such as dichloromethane.

Those skilled in the art will appreciate that it may be necessary to protect certain groups. Suitable protecting groups and methods for their attachment and removal are conventional in the art of organic chemistry, such as those described in Greene T. W. ‘Protective groups in organic synthesis’ New York, Wiley (1981).

Compounds of formulae (II) and (III) are commercially available or may be prepared according to known methods or analogous to known methods.

Pharmaceutically acceptable salts may be prepared conventionally by reaction with the appropriate acid or acid derivative.

Compounds of formula (I) and their pharmaceutically acceptable salts have 5HT₆ receptor antagonists are believed to be of potential use in the treatment of certain CNS disorders such as anxiety, depression, epilepsy, obsessive compulsive disorders, migraine, Alzheimers disease and enhancement of cognitive memory, sleep disorders (including disturbances of Circadian Rhythym), feeding disorders such as anorexia and bulimia, panic attacks, withdrawal from drug abuse such as cocaine, ethanol, nicotine and benzodiazepines, schizophrenia, and also disorders associated with spinal trauma and/or head injury such as hydrocephalus. Compounds of the invention are also expected to be of use in the treatment of certain GI disorders such as IBS

Thus the invention also provides a compound of formula (I) or a pharmaceutically acceptable salt thereof, for use as a therapeutic substance, in particular in the treatment or prophylaxis of the above disorders.

The invention further provides a method of treatment or prophylaxis of the above disorders, in mammals including humans, which comprises administering to the sufferer a therapeutically effective amount of a compound of formula (I) or a pharmaceutically acceptable salt thereof.

In another aspect, the invention provides the use of a compound of formula (I) or a pharmaceutically acceptable salt thereof in the manufacture of a medicament for the treatment or prophylaxis of the above disorders.

The present invention also provides a pharmaceutical composition, which comprises a compound of formula (I) or a pharmaceutically acceptable salt thereof, and a pharmaceutically acceptable carrier.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 0.05 to 20.0 mg, for example 0.2 to 5 mg; and such unit doses may be administered more than once a day, for example two or three a day, so that the total daily dosage is in the range of about 0.5 to 100 mg; and such therapy may extend for a number of weeks or months.

When administered in accordance with the invention, no unacceptable toxicological effects are expected with the compounds of the invention.

The following Examples illustrate the preparation of compounds of the invention.

### EXAMPLE 1

### N-[4-Methoxy-3-(4-methylpiperazin-1-yl)phenyl]-N-methyl-4-phenylbenzamide

A solution of biphenyl-4-carboxylic acid chloride in acetone (2 ml) was added to a solution of N-[4-methoxy-3-(4-methylpiperazin-1-yl)phenyl]-N-methylamine (1 equivalent) in acetone and the mixture stood overnight at room temperature. The resultant crystalline solid was filtered off and washed with acetone, then diethyl ether, to afford the title compound as the hydrochloride salt. MS: m/z=416 (MH⁺).

The following compounds were prepared in a similar manner from an N-alkyl-N-[4-methoxy-3-(4-methylpiperazin-1-yl)phenyl]amine and the appropriate carboxylic acid chloride:

| | MS (MH⁺) |
|---|---|
| 4-Bromo-N-ethyl-N-[4-methoxy-3-(4-methyl- | 446/443 |
| piperazin-1-yl)phenyl]-3-methylbenzamide (E2) | |
| 4-Bromo-N-[4-methoxy-3-(4-methylpiperazin-1-yl) | 460/462 |
| phenyl]-3-methyl-N-propylbenzamide (E3) | |
| 4-Bromo-N-[4-methoxy-3-(4-methylpiperazin-1-yl) | 432/434 |
| phenyl]-3-methyl-dimethylbenzamide (E4) | |
| Naphthalene-2-carboxylic acid N-[4-methoxy-3-(4-methyl- | 390 |
| piperazin-1-yl)phenyl]-N-methyl amide (E5) | |
| 3-Chlorobenzo[b]thiophene-2-carboxylic acid | 430/432 |
| N-[4-methoxy-3-(4- methylpiperazin-1-yl)phenyl]-N-methyl | |
| amide (E6) | |
| 3-Bromo-N-[4-methoxy-3-(4-methylpiperazin-1-yl)phenyl]-N- | 418/420 |
| methylbenzamide (E7) | |
| 3,4-Dichloro-N-[4-methoxy-3-(4-methylpiperazin-1-yl) | 408/410 |
| phenyl]-N-methylbenzamide (E8) | |
| 3-Bromothiophene-2-carboxyiic acid N-[4-methoxy-3-(4- | 424/426 |
| methylpiperazin-1-yl)phenyl]-N-methyl amide (E9) | |
| 4-tert Butyl-N-[4-methoxy-3-(4-methylpiperazin-1-yl) | 396 |
| phenyl]-N-methylbenzarnide (E10) | |
| 4-Bromo-N-[4-methoxy-3-(4-methylpiperazin-1-yl)phenyl]-N- | 418/420 |
| methylbenzamide (E11) | |

### EXAMPLE 12

### 3,4-Dichloro-N-(4-methoxy-3-piperazin-1-ylphenyl)-N-methylbenzamide (E12)

A solution of 1-chloroethylchloroformate (1.12 mmol), 3,4-dichloro-N-[4-methoxy-3-(4-methylpiperazin-1-yl)phenyl]-N-methylbenzamide (E8) (0.22 mmol) and diisopropylethylamine (1.14 mmol) in 1,2-dichloroethane (2 ml) was refluxed for 12 h. The solution was concentrated to a residue which was re-dissolved in methanol and refluxed for 6 h. The mixture was concentrated, and the residue partitioned between dichloromethane and aqueous sodium bicarbonate solution. The organic layer was dried, concentrated to a residue and purified by column chromatography on silica gel using a methanol/dichloromethane solvent gradient. The hydrochloride salt of the title compound (E12) was prepared by dissolving the pure material from chromatography in acetone/dichloromethane and acidifying with ethereal HCl. MH⁺ 393/395/397.

### EXAMPLE 13

### 3-Chlorobenzo[b]thiophene-2-carboxylic acid N-(4-methoxy-3-piperazin-1-ylphenyl)-N-methyl amide (E13)

The title compound was prepared from 3-chlorobenzo[b]thiophene-2-carboxylic acid [4-methoxy-3-(4-methylpiperazin-1-yl)phenyl]methyl amide (E6) according to the method described for Example 12. MH⁺ 415/417.

Method for assay of 5-HT6 antagonistic activity:

The test compounds were dissolved in polyethylene glycol:dimethyl sulphoxide (1:1) at 1 or 10 mM and diluted to 0.1 mM using 5 mM tris buffer (pH 7.7 @ 25° C.). Dissolution was assisted by addition of 0.02 ml 5M HCl plus heating to 40° C. and sonication for 10 minutes. Serial dilutions of drugs in the same buffer were carried out using either a TECAN 5052 or Biomek 2000 Workstation. Samples of the diluted test compounds (0.05 ml) were mixed with 0.05 ml of radio-ligand [³H]-LSD prepared in the incubation buffer, and 0.4 ml of a suspension of a preparation of the washed membranes of HeLa_{—}5HT6 cells (acquired from Dr. D. Sibley, NIH, Bethesda, see Ref 1)(see Table 1), also in the incubation buffer. The details of the incubation conditions for each assay are shown in Table 2. The incubation buffer was 50 mM Trizma (Sigma, UK) pH7.7 @ 25° C., 4 mM MgCl₂.

After incubation at 37° C., the mixtures were filtered using a Packard Filtermate in Packard TopCount format. Filters were washed with 4×1 ml aliquots of ice-cold incubation buffer. Filters were dried and impregnated with 0.04 ml of Microscint 20 (Packard). IC₅₀ values were estimated from the counts per minute using a four parameter logistic curve fit within EXCEL (2). Kᵢ values were calculated using the method of Cheng and Prusoff (3). pIC₅₀ and pKᵢ are the negative log10 of the molar IC₅₀ and Kᵢ respectively.

TABLE 1
Details of the methods used to prepare membranes for binding assays

| 1st resu- | spin/resu- | Incubation | protein conc. | cells/ml |
|---|---|---|---|---|
| spension | spension | before final | in stored | in stored |
| cells/ml | 1, 2 ,3 | spin | aliquots | aliquots |
| 7 × 10⁷ | Yes | 20 min at 37° C. | 4 mg/ml | 1.0 × 10⁸ |

TABLE 2
Summary of receptor binding assay conditions

| protein | radio-ligand | Specific | Non- | |
|---|---|---|---|---|
| (ug/ | [³H]-LSD | Activity | Specific | |
| sample) | (nM) | (Ci/mmol) | Definition | Kd (nM) |
| 40 | 2.0 | 83 | Methiothepin | 3.1 |

References

1. MONSMA, F. J., SHEN, Y., WARD, R. P., HAMBLIN, M. W., SIBLEY, D. R. 1993. Cloning and expression of a novel serotonin receptor with high affinity for tricyclic psychotropic drugs. Mol. Pharmacol., 43, 320-327.

2. BOWEN, W. P., JERMAN, J. C. 1995. Nonlinear regression using spreadsheets. Trends in Pharmacol. Sci., 16, 413-417.

3. CHENG, Y. C., PRUSSOF, W. H. 1973. Relationship between inhibition constant (Ki) and the concentration of inhibitor which causes 50% inhibition (IC50) of an enzymatic reaction. Biochem. Pharmacol., 92, 881-894.

The compounds of Examples all showed good selective 5-HT6 receptor antagonist activity, having pKi values above 7.0 at human cloned 5-HT6 receptors.

1. A compound of formula (I) or a salt thereof: 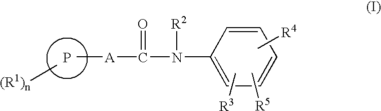
wherein:
P is phenyl, naphthyl, a bicyclic heterocyclic ring or is a 5 to 7-membered heterocyclic ring each containing 1 to 4 heteroatoms selected from oxygen, nitrogen or sulphur;
A is a single bond, a C₁₋₆alkylene or a C₁₋₆alkenylene group;
R¹ is halogen, C₁₋₆alkyl optionally substituted by one or more halogen atoms, C₃₋₆cycloalkyl, COC₁₋₆alkyl, C₁₋₆alkoxy, OCF₃, hydroxy, hydroxyC₁₋₆alkyl, hydroxyC₁₋₆alkoxy, C₁₋₆alkoxyC₁₋₆alkoxy, C₁₋₆alkanoyl, nitro, amino, C₁₋₆alkylamino or di C₁₋₆alkylamino, cyano or R¹ is phenyl, naphthyl, a bicyclic heterocyclic ring or is a 5 to 7-membered heterocyclic ring each containing 1 to 4 heteroatoms selected from oxygen, nitrogen or sulphur;
n is 0, 1, 2, 3, 4, 5 or 6:
R² is hydrogen, C₁₋₆ alkyl or aryl C₁₋₆ alkyl;
R³ is a group R⁵ or together with R⁵ forms a group (CH₂)₂O or (CH₂)₃O or R³ is linked to R² to form a group (CH₂)₂ or (CH₂)₃;
R⁴ is —X(CH₂)ₚ—R⁶ where X is a single bond, CH₂, O, NH or N—C₁₋₆alkyl and p is 0 to 6 and R⁶ is an optionally substituted 5- to 7-membered heterocyclic ring containing 1 to 3 heteroatoms selected from nitrogen, sulphur or oxygen, or R⁶ is NR⁷R⁸ where R⁷ and R⁸ are independently hydrogen, C₁₋₆ alkyl or aryl C₁₋₆ alkyl; and
R⁵ is hydrogen, halogen, C₁₋₆alkyl, C₃₋₆cycloalkyl, COC₁₋₆alkyl, C₁₋₆alkoxy, hydroxy, hydroxyC₁₋₆alkyl, hydroxyC₁₋₆alkoxy, C₁₋₆alkoxyC₁₋₆alkoxy, C₁₋₆alkanoyl, nitro, trifluoromethyl, cyano or aryl.

2. A compound according to claim 1 in which P is phenyl.

3. A compound according to any one of claims 1 to 2 in which R² is C₁₋₆alkyl.

4. A compound according to any one of claims 1 to 3 in which R⁴ is an optionally substituted piperazine ring.

5. A compound according to any one of claims 1 to 4 in which R⁵ is C₁₋₆alkoxy.

6. A compound according to any one of claims 1 to 5 in which n is 1 or 2.

7. A compound according to claim 1 which is:
N-[4-Methoxy-3-(4-methylpiperazin-1-yl)phenyl]-N-methyl-4-phenylbenzamide,
4-Bromo-N-ethyl-N-[4-methoxy-3-(4-methylpiperazin-1-yl)phenyl]-3-methylbenzamide,
4-Bromo-N-[4-methoxy-3-(4-methylpiperazin-1-yl)phenyl]-3-methyl-N-propylbenzamide,
4-Bromo-N-[4-methoxy-3-(4-methylpiperazin-1-yl)phenyl]-3,N-dimethylbenzamide,
Naphthalene-2-carboxylic acid N-[4-methoxy-3-(4-methylpiperazin-1-yl)phenyl]-N-methyl amide,
3-Chlorobenzo[b]thiophene-2-carboxylic acid-N-[4-methoxy-3-(4-methylpiperazin-1-yl)phenyl]-N-methyl amide,
3-Bromo-N-[4-methoxy-3-(4-methylpiperazin-1-yl)phenyl]-N-methylbenzamide,
3,4-Dichloro-N-[4-methoxy-3-(4-methylpiperazin-1-yl)phenyl]-N-methylbenzamide,
3-Bromothiophene-2-carboxylic acid-N-[4-methoxy-3-(4-methylpiperazin-1-yl)phenyl]-N-methyl amide,
4-tert Butyl-N-[4-methoxy-3-(4-methylpiperazin-1-yl)phenyl]-N-methylbenzamide,
4-Bromo-N-[4-methoxy-3-(4-methylpiperazin-1-yl)phenyl]-N-methylbenzamide,
3,4-Dichloro-N-(4-methoxy-3-piperazin-1-ylphenyl)-N-methylbenzamide, or
3-Chlorobenzo[b]thiophene-2-carboxylic acid N-(4-methoxy-3-piperazin-1-ylphenyl)-N-methyl amide,
and pharmaceutically acceptable salts thereof.

8. A compound according to any one of claims 1 to 7 for use in therapy.

9. A compound according to any one of claims 1 to 7 for use in therapy, in which the beneficial activity is effected by antagonism of 5-HT6 receptors.

10. A compound according to any one of claims 1 to 7 for use in the treatment of schizophrenia, Alzheimer's disease and/or depression.

11. A pharmaceutical composition which comprises a compound according to any one of claims 1 to 7 and a pharmaceutically acceptable carrier or excipient.

12. A process for the preparation of a compound of formula (I) or a salt thereof 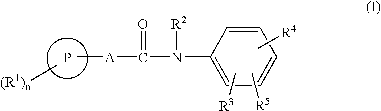
wherein:
P is phenyl, naphthyl, a bicyclic heterocyclic ring or is a 5 to 7-membered heterocyclic ring each containing 1 to 4 heteroatoms selected from oxygen, nitrogen or sulphur;
A is a single bond, a C₁₋₆alkylene or a C₁₋₆alkenylene group;
R¹ is halogen, C₁₋₆alkyl optionally substituted by one or more halogen atoms, C₃₋₆cycloalkyl, COC₁₋₆alkyl, C₁₋₆alkoxy, OCF₃, hydroxy, hydroxyC₁₋₆alkyl, hydroxyC₁₋₆alkoxy, C₁₋₆alkoxyC₁₋₆alkoxy, C₁₋₆alkanoyl, nitro, amino, alkylamino or dialkylamino, cyano or R¹ is phenyl, naphthyl, a bicyclic heterocyclic ring or is a 5 to 7-membered heterocyclic ring each containing 1 to 4 heteroatoms selected from oxygen, nitrogen or sulphur;
n is 0, 1, 2, 3, 4, 5 or 6:
R² is hydrogen, C₁₋₆ alkyl or aryl C₁₋₆ alkyl;
R³ is a group R⁵ or together with R⁵ forms a group (CH₂)₂O or (CH₂)₃O or R³ is linked to R² to form a group (CH₂)₂ or (CH₂)₃;
R⁴ is —X(CH₂)p-R⁶ where X is a single bond, CH₂, O, NH or N—C₁₋₆alkyl and p is 0 to 6 and R⁶ is an optionally substituted 5- to 7-membered heterocyclic ring containing 1 to 3 heteroatoms selected from nitrogen, sulphur or oxygen, or R⁶ is NR⁷R⁸ where R⁷ and R⁸ are independently hydrogen, C₁₋₆ alkyl or aryl C₁₋₆ alkyl; and
R⁵ is hydrogen, halogen, C₁₋₆alkyl, C₃₋₆cycloalkyl, COC₁₋₆alkyl, C₁₋₆alkoxy, hydroxy, hydroxyC₁₋₆alkyl, hydroxyC₁₋₆alkoxy, C₁₋₆alkoxyC₁₋₆alkoxy, C₁₋₆alkanoyl, nitro, trifluoromethyl, cyano or aryl;
which process comprises the coupling of a compound of formula (II): 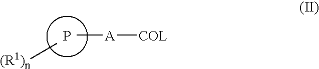
in which R¹, n, P, and A are as defined in formula (I) or protected derivatives thereof and L is a leaving group with a compound of formula (III): 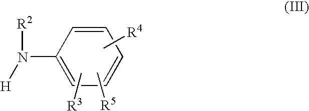
in which R², R³, R⁴ and R⁵ are as defined in formula (I) or protected derivatives thereof and optionally thereafter:
removing any protecting groups,
forming a pharmaceutically acceptable salt.
